(19) United States
(12) Patent Application Publication
(10) Pub. No.: US 2012/0219981 A1
(43) Pub. Date: Aug. 30, 2012

(54) MECHANICAL STRETCHING DEVICE

(76) Inventors: Manimaran MUTHIAH, Singapore (SG); Ellen Birgitte LANE, (US)

(21) Appl. No.: 13/217,199

(22) Filed: Aug. 24, 2011

(30) Foreign Application Priority Data
Aug. 24, 2010 (SG) 201006166-1

(51) Int. Cl. C12Q 1/02; C12M 1/34

(52) U.S. Cl. 435/29; 435/287.9

(57) ABSTRACT

The present invention is directed to a mechanical stretching device, comprising a stretchable substrate comprising at least one stretching material area within which a stretching material is placeable, and two engagement areas being located at opposite ends of the stretchable substrate, respectively, two movable elements, each of which comprising an engagement portion, wherein each of the engagement portions is capable of engaging with one of the engagement areas, and two motors, each of which being configured to drive one of the movable elements, wherein the movable elements are movable by the motors such that the engagement portions cause, after having engaged with the engagement areas, either one end or both ends of the stretchable substrate to be stretched, wherein the ends of the stretchable substrate are to be stretched along opposite directions with respect to each other.

### CROSS-REFERENCE TO RELATED APPLICATIONS

This application claims the benefit of Singapore Patent Application No. 201006166-1, filed on Aug. 24, 2010, the entire contents of which are incorporated by reference.

### TECHNICAL FIELD

Various embodiments relate to the field of mechanical stretching devices, in particular mechanical stretching devices for cell stretching.

### BACKGROUND

Cellular responses to mechanical stimuli are regulated by interaction with the extra-cellular matrix (ECM), which in turn are strongly influenced by the degree of cell stiffness. Mechanical stress exerted at cell-substrate and cell-cell interfacial boundaries is involved in the regulation of a variety of physiological process. Living cells can sense mechanical forces and convert them into biological responses. Similarly, biological and biochemical signals can influence the abilities of cells to sense, generate and bear mechanical forces.

Cells in tissues are living in differently stiff environments and are subjected to different types of mechanical load; such mechanical cues determine cell fate, phenotype and behavior. Blood circulating cells experience fluid flow shear stress cells residing in bone and cartilage are under compressive load and a number of cell types are subjected to stretch. Cardiac myocytes and endothelial and smooth muscle cells of vessels of the intestine and of the airways undergo cyclic stretch. Other cells types, including skeletal muscle cells, connective tissue fibroblasts and epidermal keratinocytes bear gradual stretches of different degrees and of varying rates.

By including a mechanical component, such as stretch and matrix elasticity, significantly improvements on the physiological relevance of cell culture studies of cell functions have been observed. Moreover, stretchable substrates are important tools to study the mechanisms of cell mechanosensing and the consequences of mechanical protein deformation.

Studies into the mechanics of cells, sub-cellular components and biological molecules have rapidly evolved during the past decade with significant implications for biotechnology and human health. Adhesion of cells to extracellular matrix (ECM) through focal adhesion complexes provides both signaling and structural functions. While many tools exist to stretch cells mechanically to manipulate the biochemical adhesiveness of experimental substrates, relatively few approaches have been developed to engineer the stretching substrate and the stretching orientation with high frequency and higher amplitude in order to study the full impact of the mechanical stress on the live cells.

In the case of epithelial cells such as skin, it is becoming increasingly apparent that epithelial cell movement and changes in morphology are central to both development and regeneration of epithelial organs. For example, keratins which are intermediate filaments in the keratinocytes join together and form highly resilient fibers in the lower portion of skin, helping make it durable. If the keratin are defective, they do not mesh and the lower skin tissue becomes unusually fragile and gets damaged from the mildest mechanical stress; thereby leading to blistering pain, a higher risk of infection, and in the most severe cases, death.

Epidermolysis bullosa (EB) is a group of inherited bullous disorders characterized by blister formation in response to mechanical trauma. EB subtypes have been classified according to skin morphology. The molecular basis of EB has led to the development of diagnostic tools, including prenatal and pre-implantation testing. Based on a better understanding of the basement membrane zone (BMZ) and the genes responsible for its components, new treatments (e.g., gene or protein therapy) may provide solutions to the skin fragility found in patients with EB.

Thus, it is an object to provide a new cell stretching system and method in order to apply the mechanical stress directly to the skin cells and study the effect of this stress on these cells, for example, to better understand the EB disease.

### SUMMARY

In a first aspect, the present invention relates to a mechanical stretching device, comprising a stretchable substrate comprising at least one stretching material area within which a stretching material is placeable, and two engagement areas being located at opposite ends of the stretchable substrate, respectively, two movable elements, each of which comprising an engagement portion, wherein each of the engagement portions is capable of engaging with one of the engagement areas, and two motors, each of which being configured to drive one of the movable elements, wherein the movable elements are movable by the motors such that the engagement portions cause, after having engaged with the engagement areas, either one end or both ends of the stretchable substrate to be stretched, wherein the ends of the stretchable substrate are to be stretched along opposite directions with respect to each other.

According to a second aspect, the present invention relates to a stretching device, comprising a stretchable substrate area being configured to receive a stretchable substrate comprising at least one stretching material area within which a stretching material is placeable, and two engagement areas being located at opposite ends of the stretchable substrate, respectively, two movable elements, each of which comprising an engagement portion, wherein each of the engagement portions is capable of engaging with one of the engagement areas, and two motors, each of which being configured to drive one of the movable elements, wherein the movable elements are movable by the motors such that, when the stretchable substrate is placed within the stretchable substrate placing area, the engagement portions cause, after having engaged with the engagement areas, either one end or both ends of the stretchable substrate to be stretched, wherein the ends of the stretchable substrate are stretchable along opposite directions with respect to each other.

According to a third aspect, the present invention relates to a method of carrying out a stretch test with live cells, the method comprising placing and cultivating live cells on the stretching material of the stretching device.

### BRIEF DESCRIPTION OF THE DRAWINGS

In the drawings, like reference characters generally refer to the same parts throughout the different views. The drawings are not necessarily to scale, emphasis instead generally being placed upon illustrating the principles of the invention. In the following description, various embodiments of the invention are described with reference to the following drawings, in which:

FIG. 1 shows a schematic block diagram of a mechanical stretching device, according to various embodiments;

FIG. 2 shows a schematic block diagram of a mechanical stretching device, according to various embodiments;

FIG. 3 shows a schematic block diagram of a mechanical stretching device, according to various embodiments;

FIG. 4 shows a flow chart illustrating a method of a method of carrying out a stretch test with live cells, according to various embodiments;

FIG. 5 shows a schematic diagram of an exemplary cell stretching system;

FIG. 6 shows a schematic diagram of an exemplary multi-well cell culture device;

FIG. 7 shows a CAD design illustrating a perspective view of an exemplary cell stretching system;

FIG. 8 shows a photo image of an exemplary prototype of a mechanical cell stretching system;

FIG. 9 shows a photo image of various type of exemplary fabricated culture substrates;

FIG. 10 shows a plot of force vs elongation (tensile strength) of an exemplary fabricated device;

FIG. 11 shows optical microscope images of a PDMS surface (a) before polishing; (b) after polishing; and (c) then coating with Collagen-IV;

FIG. 12 shows optical microscope images of (a) the NEB1, and (b) the mutant NEB 1 cells cultured on a surface of a coated PDMS stretching device;

FIG. 13 shows a photo image of an experimental setup;

FIG. 14 shows a snap-shot view of a control software menu displaying a stretching graph of strength length vs time;

FIG. 15 shows phase contrast images of wild-type NEB1.K14GFP cells which are grown on the fabricated device (a) before stretch, and (b) after subjecting to 30 mins of mechanical stress using the I-MCS1 system;

FIG. 16 shows phase contrast images of a separate sample of wild-type NEB1.K14GFP cells which are grown on the fabricated device on which mechanical stretch is carried out at a frequency of 2 Hz and an amplitude of 50% for times varying up to 180 mins (more specifically, (a) at 0 min, (b) after 15 mins of stretching, (c) after 30 mins of stretching, (d) after 1 hour of stretching, (e) after 2 hours of stretching, and (f) after 3 hours of stretching) at 37° C. and 5% CO₂;

FIG. 17 shows phase contrast images of mutant NEB1.K14GFP cells which are grown on the fabricated device (a) before stretch, and (b) after subjecting to 2 hours of mechanical stress using the I-MCS1 system;

FIG. 18 shows phase contrast images of a separate sample of mutant NEB1.K14GFP cells which are grown on the fabricated device on which mechanical stretch is carried out at a frequency of 2 Hz and an amplitude of 50% for times varying up to 180 mins (more specifically, (a) at 0 min, (b) after 15 mins of stretching, (c) after 30 mins of stretching, (d) after 1 hour of stretching, (e) after 2 hours of stretching, and (f) after 3 hours of stretching) at 37° C. and 5% CO₂;

FIG. 19 shows optical microscope images of a well-formed network of keratin filaments in the control wild-type NEB1.K14GFP cells (a) before stretch, where filaments are intact, and (b) after 2 hours of stretching at 4 Hz;

FIG. 20 shows optical microscope images of a well-formed network of keratin filaments in the mutant NEB1.R125P.K14GFP cells (a) before stretch, and (b) after 5 mins of stretching at 4 Hz; and

FIG. 21 shows fluorescence microscope images of the breaking down of the cell junction of wild-type NEB1.K14GFP cells, when applied with a mechanical stress for (a) 0 min, (b) 1 hour, and (c) 2 hours at 4 Hz with an amplitude of 50%.

### DETAILED DESCRIPTION

The following detailed description refers to the accompanying drawings that show, by way of illustration, specific details and embodiments in which the invention may be practiced. These embodiments are described in sufficient detail to enable those skilled in the art to practice the invention. Other embodiments may be utilized and structural, logical, and electrical changes may be made without departing from the scope of the invention. The various embodiments are not necessarily mutually exclusive, as some embodiments can be combined with one or more other embodiments to form new embodiments.

In order that the invention may be readily understood and put into practical effect, particular embodiments will now be described by way of examples and not limitations, and with reference to the figures.

FIG. 1 shows a schematic block diagram of a mechanical stretching device, according to various embodiments. In a first aspect, various embodiments provide a mechanical stretching device 100 comprising a stretchable substrate 102 comprising at least one stretching material area 104 within which a stretching material 106 is placeable, and two engagement areas 108 being located at opposite ends of the stretchable substrate 102, respectively, two movable elements 110, each of which comprising an engagement portion 112, wherein each of the engagement portions 112 is capable of engaging with one of the engagement areas 108, and two motors 114, each of which being configured to drive one of the movable elements 110, wherein the movable elements 110 are movable by the motors 114 such that the engagement portions 112 cause, after having engaged with the engagement areas 108, either one end or both ends of the stretchable substrate 102 to be stretched, wherein the ends of the stretchable substrate 102 are to be stretched along opposite directions with respect to each other.

In various embodiments, the movable elements 110 are independently movable by the motors 114 such that the ends of the stretchable substrate 102 are independently stretchable. The motors 114 may be motors which produce none or only little heat when in operation, in particular to maintain a physiological temperature. For example, the motors 114 may be voice coil motors. Voice-coil motors may provide a smooth pulling of the stretching device. In the context of various embodiments, the term “only little heat” may generally refer to an amount of heat that raises the temperature measured by the thermocouple by about 1 degree Celsius. The term “physiological” may generally refer to being consistent with the normal functioning of an organism. For example, the physiological temperature may refer to the body temperature.

In various embodiments, the motors 114 may be drivable such that the stretchable substrate 102 is continuously and/or periodically stretched and/or de-stretched. In the context of various embodiments, the term “continuously” may refer to as being uninterrupted and without cessation. The term “periodically” may refer to as being continually, and/or recurring at intervals of time. The term “de-stretched” may refer to being in the absence of a stretching influence or being released from a stretched configuration.

In one embodiment, the motors 114 may be drivable such that a stretching frequency or a de-stretching frequency of the stretchable substrate 102 ranges from 0.5 Hz to 10 Hz or from 4 Hz to 10 Hz or from 0.5 Hz to 4 Hz or is 0.5 Hz or is 4 Hz or is 10 Hz. For example, the frequency or de-stretching frequency may be selected from the ranges of 1 Hz to 10 Hz, 2 Hz to 10 Hz, 3 Hz to 10 Hz, 5 Hz to 10 Hz, 6 Hz to 10 Hz, 7 Hz to 10 Hz, 8 Hz to 10 Hz, 9 Hz to 10 Hz, 1 Hz to 9 Hz, 2 Hz to 9 Hz, 3 Hz to 9 Hz, 4 Hz to 9 Hz, 5 Hz to 9 Hz, 6 Hz to 9 Hz, 7 Hz to 9 Hz, 8 Hz to 9 Hz, 1 Hz to 8 Hz, 2 Hz to 8 Hz, 3 Hz to 8 Hz, 4 Hz to 8 Hz, 5 Hz to 8 Hz, 6 Hz to 8 Hz, 7 Hz to 8 Hz, 1 Hz to 7 Hz, 2 Hz to 7 Hz, 3 Hz to 7 Hz, 4 Hz to 7 Hz, 5 Hz to 7 Hz, 6 Hz to 7 Hz, 1 Hz to 6 Hz, 2 Hz to 6 Hz, 3 Hz to 6 Hz, 4 Hz to 6 Hz, 5 Hz to 6 Hz, 1 Hz to 5 Hz, 2 Hz to 5 Hz, 3 Hz to 5 Hz, 4 Hz to 5 Hz, 1 Hz to 4 Hz, 2 Hz to 4 Hz, 3 Hz to 4 Hz, 1 Hz to 3 Hz, and 2 Hz to 3 Hz.

In various embodiments, the movement of the movable elements 110 may be controlled such that the stretchable substrate 102 is stretched up to 100% in its length. In the context of various embodiments, the phrase “stretched up to 100% in its length” may generally refer to being stretched or extended to its full length without overstretching its maximum limits. The movement of the movable elements 110 may be controlled such that the stretchable substrate 102 may stretched up to 95% or 90% or 85% or 80% or 75% or 70% in its length.

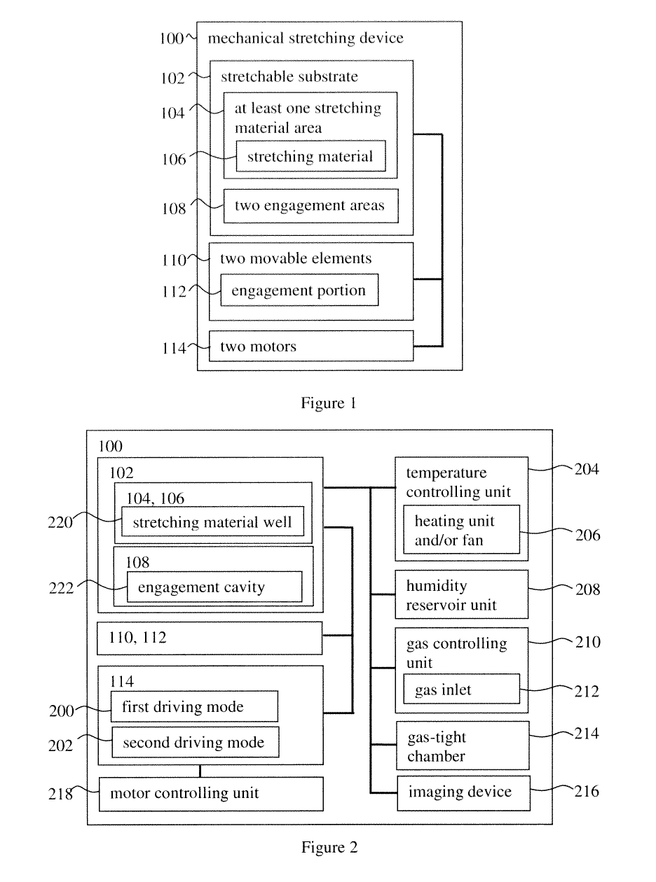

In FIG. 2, various embodiments provide the stretching device 100, wherein the motors 114 are switchable between two driving modes, wherein, in a first driving mode 200, the stretchable substrate 102 is caused to be stretched in opposite directions, whereas, in a second driving mode 202, the stretchable substrate 102 is caused to be stretched in only one direction.

Various embodiments provide the stretching device 100 further comprising a temperature controlling unit 204 which controls the temperature of the stretchable substrate 102 and its micro-environment to a predetermined temperature value. In the context of various embodiments, the term “micro-environment” may generally refer to a small specific area, in this case, surrounding the stretchable substrate. For example, this small specific area may refer to an area covered by a radius of about 10 μm.

The temperature controlling unit 204 may comprise a heating unit and/or a fan 206 in particular for the uniform distribution of heat and to maintain the temperature.

In various embodiments, the stretching device 100 may further comprise a humidity reservoir unit 208 which controls or maintains the humidity of the environment of the stretchable substrate 102 to a predetermined humidity value. The stretching device 100 may further comprise a gas controlling unit 210 which controls gas parameters of a gas surrounding the stretchable substrate 102 to predetermined gas parameters. In one embodiment, the gas controlling unit 210 may comprise a gas inlet 212 adapted to supply gas to the surrounding of the stretchable substrate 102.

In various embodiments, the stretching device 100 may further comprise a gas-tight chamber 214 surrounding the stretchable substrate 102, wherein the movable elements 110 extend from outside of the chamber 214 through walls of the chamber 214 into the inside of the chamber 214, and wherein temperature, humidity and gas parameters within the chamber 214 are controlled by the temperature controlling unit 204, and the gas controlling unit 210.

In various embodiments, the stretching device 100 may further comprise an imaging device 216 adapted to image the stretching material 106 during the stretching of the stretchable substrate 102. For example, the imaging device 216 may comprise a microscope base plate. The imaging device 216 may comprise a commercially available microscope.

In various embodiments, the stretching device 100 may further comprise a motor controlling unit 218 which controls the operation of the motors 114 in a fully automated manner.

In various embodiments, the stretchable substrate 102 may comprise, within the stretching material area 104, at least one stretching material well 220 into which the stretching material 106 is fillable.

In various embodiments, the stretchable substrate 102 may comprise, within each engagement area 108, at least one engagement cavity 222 into which the engagement portions 112 of the moving elements 110 are configured to be introduced.

In various embodiments, the stretching material 106 and/or the stretchable substrate 102 may be made of a stretchable material. For example, the stretchable material is a stretchable polymeric material. For example, the stretchable polymeric material is poly-di-methyl-siloxane (PDMS).

In various embodiments, the stretching material 106 and/or the stretchable substrate 102 may be coated with an adhesion layer facilitating binding of live cells to be analyzed in a stretch test carried out with the stretching device 100. For example, the stretching material 106 and/or the stretchable substrate 102 are coated with Matrigel or collagen, such as Collagen-IV, or fibronectin.

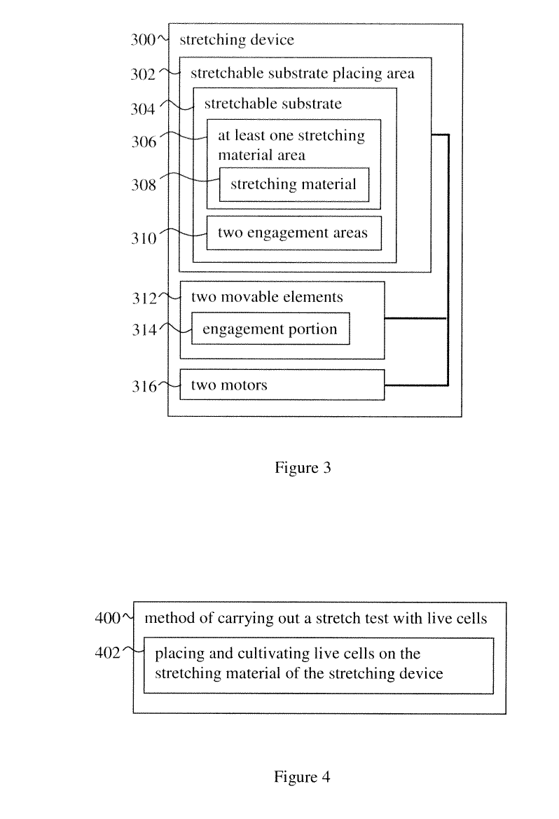

FIG. 3 shows a schematic block diagram of a mechanical stretching device, according to various embodiments. In a second aspect, various embodiments provide a stretching device 300, comprising a stretchable substrate placing area 302 being configured to receive a stretchable substrate 304 comprising at least one stretching material area 306 within which a stretching material 308 is placeable, and two engagement areas 310 being located at opposite ends of the stretchable substrate 304, respectively, two movable elements 312, each of which comprising an engagement portion 314, wherein each of the engagement portions 314 is capable of engaging with one of the engagement areas 310, and two motors 316, each of which being configured to drive one of the movable elements 312, wherein the movable elements 312 are movable by the motors 316 such that, when the stretchable substrate 304 is placed within the stretchable substrate placing area 302, the engagement portions 314 cause, after having engaged with the engagement areas 310, either one end or both ends of the stretchable substrate 304 to be stretched, wherein the ends of the stretchable substrate 304 are stretchable along opposite directions with respect to each other.

FIG. 4 shows a flow chart illustrating a method of a method of carrying out a stretch test with live cells, according to various embodiments. In a third aspect, various embodiments provide a method of carrying out a stretch test with live cells 400, comprising placing and cultivating live cells on the stretching material of the stretching device 402. In various embodiments, the stretchable substrate of the stretching device may be imaged with the imaging device while stretching the stretchable substrate. In one embodiment, the stretchable substrate of the stretching device may be imaged with the imaging device while stretching the stretchable substrate by holding the substrate in a pre-determined stretched position for a pre-determined period of time.

In various embodiments, the cells may be eukaryotic cells. The cells may also be selected from the group consisting of epithelial cells, muscle cells, cells isolated from intestines, cells isolated from airways, cells derived from bone, cells derived from cartilage and cells isolated from blood stream.

In various embodiments, the cells may be selected from the group consisting of cardiac myocytes, endothelial muscle cells, smooth muscle cells, skeletal muscle cells, connective tissue fibroblasts and epidermal keratinocytes. For example, the live cells may be wild type cells or genetically modified mutant cells.

Various embodiments provide a new mechanical cell stretching system for live cell stretching and simultaneously to monitor and image the cells under stress. Mechanical stress may be applied externally using this stretching system on the normal and mutant skin cells to study the effect of this mechanical stress on these cells under normal physiological environment. Measuring techniques to quantify the extent of mechanical stress on skin are needed for establishing successful treatment strategies to combat skin diseases such as blistering and other processes of skin degeneration.

### Experimental Section

In order to apply the mechanical stress directly to the skin cells and study the effect of this stress on these cells to better understand the EB disease, a new cell stretching system and method, which may be used to stretch the cells up to 10 Hz with the maximum stretching length of 100% is explored. By using the microfabrication technology, a multi-well stretching device based on poly-di-methylsiloxane (PDMS) may also be developed. This device may be used to culture the cells on to the PDMS substrate for stretching and simultaneously to monitor the cell behaviour and perform live-cell imaging using an inverted microscope.

Design and development of high-frequency mechanical stretching system

The first step towards designing the experiment for studying the mechanical properties of cells under stress is to design and develop a mechanical cell stretching system in order to apply the external stress on cells. To achieve this, a voice-coil based motor may be used in the stretching system instead of a stepper motor so as to minimize the heat generation during operation. The system may hold a multi-well PDMS based cell culture device with a 3-pin holder to facilitate the transfer of external force to the cell culture device equally on the both direction while stretching.

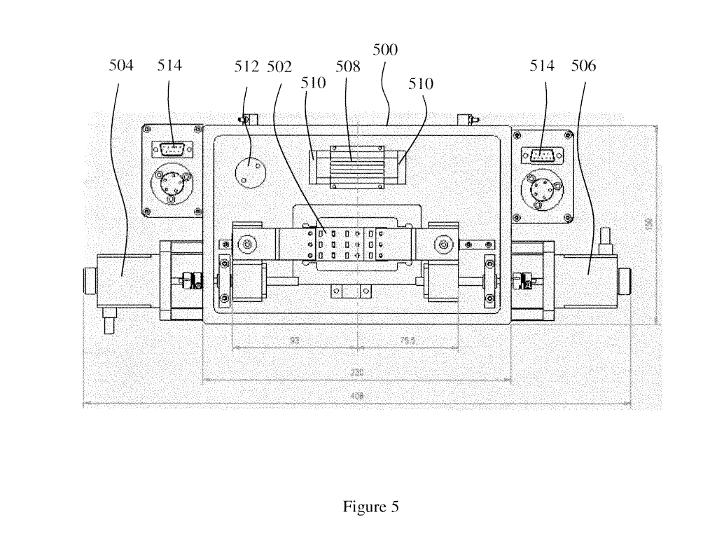

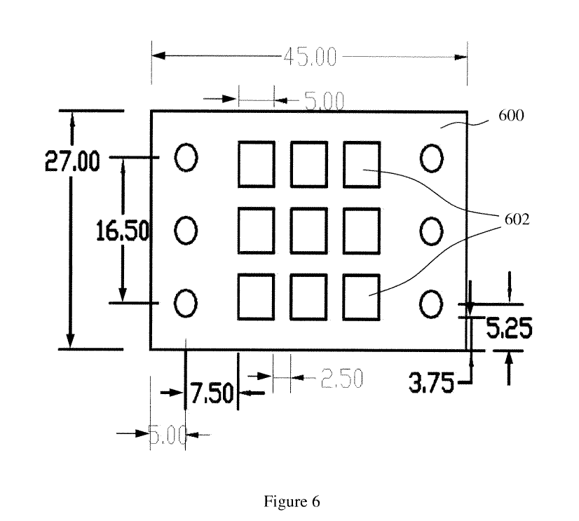

FIGS. 5 and 6 show schematic diagrams of an exemplary cell stretching system and an exemplary multi-well cell culture device, respectively.

In FIG. 5, the cell stretching system 500 comprises a cell culture device 502, motors 504, 506, an electric heating element 508, cooling fans 510, a water reservoir 512 and computer interface connections 514. Dimensions of the various components of the cell stretching system 500 are shown in FIG. 5.

In FIG. 6, the multi-well cell culture device 600 comprises an array of wells 602. Dimensions of the various parts of the multi-well cell culture device 600 are shown in FIG. 6.

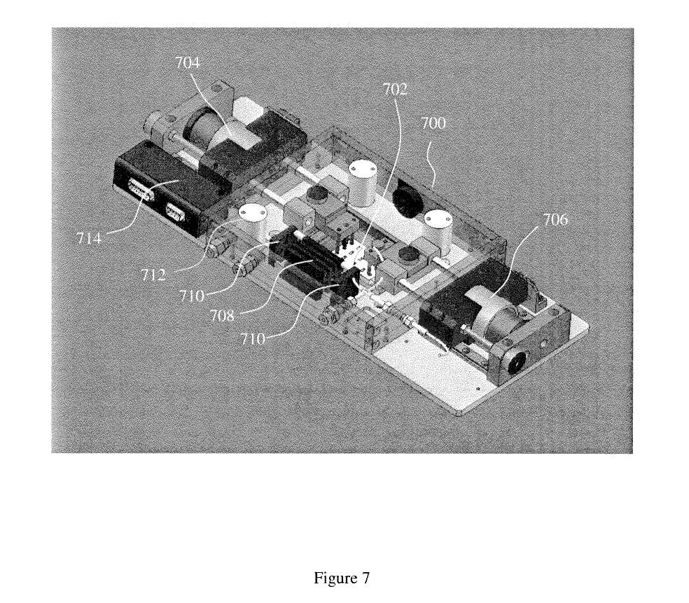

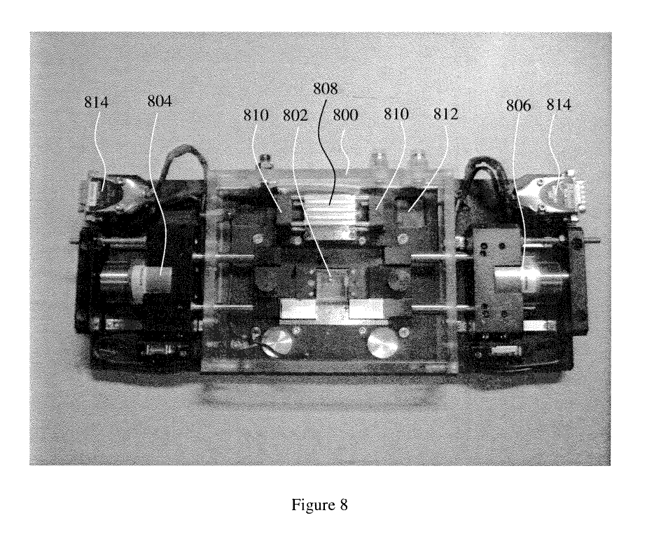

FIGS. 7 and 8 show a CAD design of an exemplary cell stretching system and a photo image of an exemplary prototype of a mechanical cell stretching system, respectively.

In FIG. 7, the CAD design of the cell stretching system 700 comprising a cell culture device 702, motors 704, 706, an electric heating element 708, cooling fans 710, a water reservoir 712 and a computer interface connections 714 is presented.

In FIG. 8, the photo image of the prototype of a mechanical cell stretching system 800 comprising a cell culture device 802, motors 804, 806, an electric heating element 808, cooling fans 810, a water reservoir 812 and a computer interface connections 814 is shown. The prototype is of a version 1 type, referred to as IMB Mechanical Cell Stretching (I-MCS1) system.

This new stretching technique using voice-coil based motor may provide the maximum frequency up to 10 Hz with minimum noise and provide a smooth pulling of a stretching device attached with the system.

There may be a built-in heating system available inside the device with a thermocouple feedback mechanism to control the temperature and to keep the system at about 37° C. throughout an experiment. A water reservoir may be available inside the stretching unit to keep the system in a moisturized condition to ensure the cells are provided with required humidity. There may be two inlets available in the system to provide the O₂ and CO₂ supply so that the physiological environment may be provided to the cells during the course of the stretching experiment. The entire system may be designed in such a way that this stretching unit may be easily fixed on any standard inverted microscopes available in the market such as the Olympus IX71 microscope, the Olympus IX81 microscope, microscopes from Zeiss and microscopes from Nikon.

The mechanical stretching may be automated and completely controlled by a computer using a Labview based software (from National Instruments Corporation, US) to set the stretching parameters and control the system accordingly. The software may be programmed based on the requirement of whether to stretch the device only in one direction while affixing/anchoring the other end; or to stretch the both side of the device simultaneously. It may also possible to perform the experiment with stretch and hold to monitor and capture the cell behavior under mechanical stress. The maximum operating frequency of the system may be set up to 10 Hz with an amplitude of up to 100% for continuous operation of up to 24 hours.

Development of PDMS based stretchable multi-well membrane devices

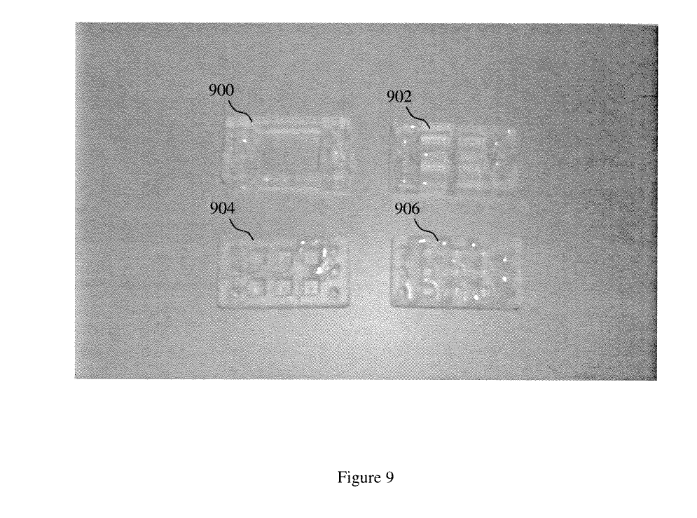

Culture substrates may be produced using PDMS silicone elastomer (Sylgard 184, Dow Corning, Wiesbaden, Germany) with 1:10 mixing ratio of curing agent-to-base. FIG. 9 shows fabricated single well 900, four-well 902, six-well 904 and nine-well 906 stretching devices. Others, for example, two-well or sixteen-well or multi-well stretching devices may also be produced. To fabricate these multi-well stretching devices, silicone elastomer is mixed with a curing agent and bubbles are removed using vacuum pump based desiccators. Once the bubbles are removed from the mixed elastomer solution, they are then poured onto the device mold and are incubated in an oven for about 4 hours at 80° C. After incubation, the mold is carefully removed and the stretching device is slowly separated from the mold (examples as seen in FIG. 9).

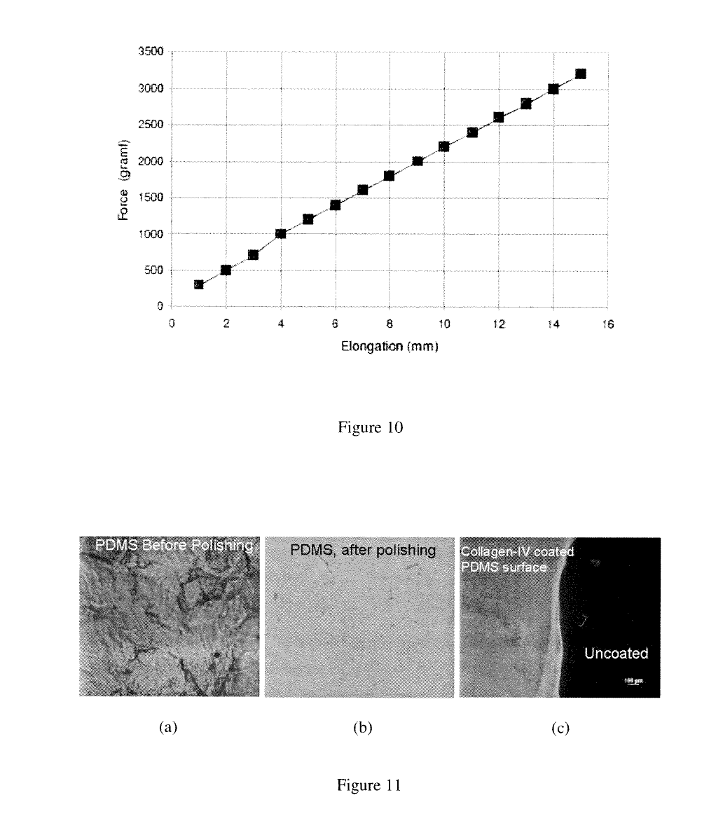

For stretching assays, PDMS membranes with a bottom thickness of 250 μm, which is stretchable up to 100% may be fabricated. FIG. 10 shows a plot of force vs elongation of a fabricated device to determine the durability of this sample. In FIG. 10, it is shown that force is directly proportional to elongation and the tensile strength of this sample is about 200 gram-force (gramf) per mm.

The pre-treated PDMS membranes are kept at 60° C. for about 1 hour and treated with plasma oxygen for 1 min at 200 mT and 50 sccm flow of oxygen in a plasma chamber (Harris Plasma, N.Y., USA) to generate free surface oxygen groups.

Comparison of IMB stretching system with other commercially available systems is shown in Table 1.

TABLE 1
Comparison of a Flexcell tension system (FX-4000 from Flexcell
International Corporation, US) and a STREX cell strain system
(ST-195M from B-Bridge International, Inc., US) with IMB stretching
system, I-MCS1.

| Parameters | Flex Cell | STREX | I-MCS1 (IMB) |
|---|---|---|---|
| Mode | Cyclic | Static | Cyclic & Static |
| Frequency | 5 Hz | 0.5 Hz | 10 Hz |
| Stretching length | 20% | 20% | 100% |
| Stretching time | 12 h | 2 h | 24 h |
| Incubation | NO | Separate | Bait-in heating with |
| | | heating coil | CO₂ supply |
| Laser Port | NO | NO | YES |
| Live cell | NO | Need to stop | YES |
| Imaging | | the system | |
| Consumable | 6-well plate | Single well | Multi-well device |
| | | device | |
| Automation | NO | NO | YES |
| Maintenance | High | Moderate | Low |

As seen in Table 1, in comparison to the Flexcell tension system and the STREX cell strain system, the I-MCS1 system advantageously provides maximum of 100% stretching length and 24 hours of stretching time. This is a significant improvement over the existing systems, which could only provide 20% stretching length and maximum of about 12 hours of stretching time. Further, real-time monitoring and live cell imaging may also be carried out using the I-MCS1 system while the other two systems would require the stretching process to be stopped before carrying out any imaging or would not be able to conduct any imaging at all.

Coating of PDMS multi-well stretching device with Matrigel/Collagen-IV

Fabricated PDMS stretching device may be polished prior to coating so that a relatively grain-boundary free smooth transparent surface may be obtained and this in turn helps to get more uniform layer of coating. FIGS. 11(a) and 11(b) show optical microscope images of the PDMS surface before and after polishing, respectively.

For the coating process, the fabricated PDMS stretching device is first pre-cooled on ice and placed on a sample holder of a spinner (Model WS 656SZ 6NPP/A1/AR1—Laurell Technologies Corporation, US). Then 200 μl of 0.12 mg/ml of Matrigel (Becton Dickinson, US) is added on the PDMS before running a spinning program. The spinning program consists of increasing steps to 40000 rpm for 30 sec and then decreasing steps to 0 rpm. The coated PDMS is then washed once in distilled water before being blow-dried with compressed nitrogen gas. The coated device is left in the 37° C. incubator for 1 hour. To determine the presence of Matrigel coating, immunofluoresence staining using the antibodies may be conducted.

To enable clearer observation, a piece of scotch tape may be applied to half of a PDMS surface before coating the entire PDMS with different concentration of Matrigel using the spinning method. This would create a coated and uncoated Matrigel surface side by side when the scotch tape is removed after the spinning process. Immunostaining is then performed on these PDMS surfaces. The coated PDMS is first blocked in 10% goat serum for 30 mins before incubating with rabbit primary antibody against Mouse Collagen IV (AB756P, Millipore, US) overnight at room temperature at a concentration of 1:50. After PBS wash, the coated PDMS is incubated for 2 hours in 1:250 diluted goat anti-rabbit secondary antibody conjugated to Alexa 488 fluorochrome (Invitrogen, US) and is subsequently imaged. FIG. 11(c) shows the contrasting images of an uncoating PDMS surface alongside a Collagen IV-coated PDMS surface.

Materials—Cell Culture on the Coated PDMS Stretching Devices

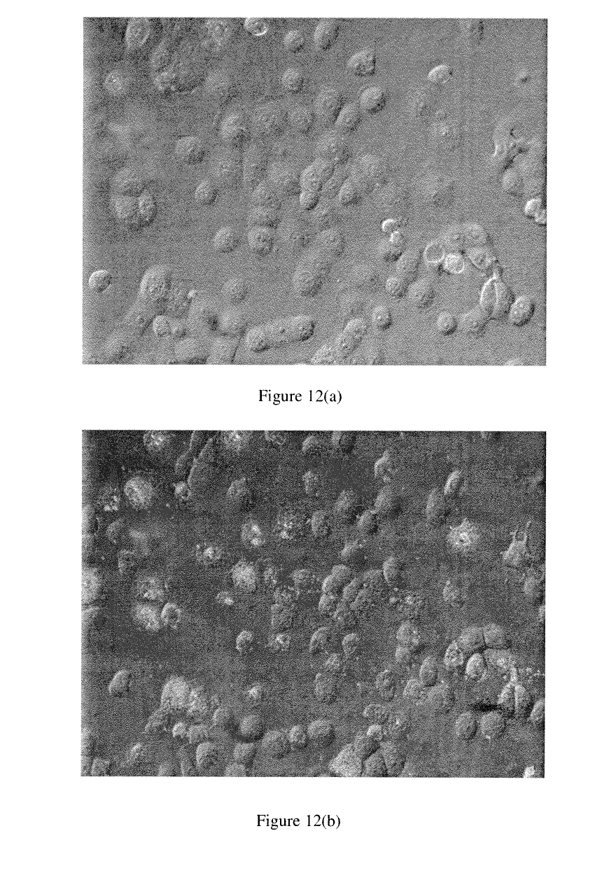

NEB1.GFP-K14.R125P and NEB1.GFP-K14.WT (a mutant NEB1 and a wild-type NEB1, respectively, from Dundee, Scotland) are maintained in standard keratinocyte tissue culture medium Dulbecco's modified Eagle's medium with 25% Ham's F12 medium, 10% fetal bovine serum, 0.4 μg mL⁻¹ hydrocortisone, 1.8×10⁻⁴ mol L⁻¹ adenine, 5 μg mL⁻¹ transferrin, 2×10⁻¹¹ mol L⁻¹ lyothyronine, 5 μg mL⁻¹ insulin, 10 ng mL⁻¹ epidermal growth factor and 1% penicillin-streptomycin. Cells are cultured at 37° C. with 5% CO₂. Cells at log phase are trypsinised and seeded on the device at the appropriate cell density, to reach 60 to 70% confluency after two to four days. Seeding density and seeding days in device may be optimized to reach desired confluency for the mechanical stretch experiment. FIG. 12 shows optical microscope images of (a) the NEB1, and (b) the mutant NEB1 cells cultured on a surface of a coated PDMS stretching device.

For the four-wells chamber, 40 000 cells of R125P and 60000 cells of WT are seeded for two days before stretch. For the single chamber, 13000 cells of R125P and 22000 cells of WT are seeded for four days before stretch. Culture mediums are replaced prior to stretch.

Method and Setup—Stretching experiment and optical microscopy for live-cell imaging.

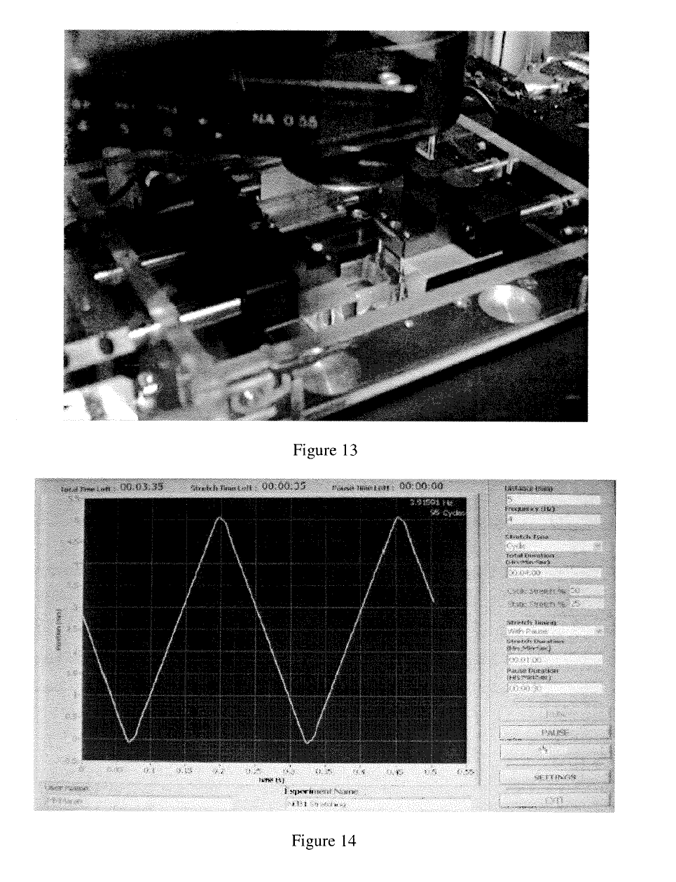

The stretching device is transferred from the tissue culture room to the microscope attached with a built-in incubator for live cell-stretching and imaging experiment. The stretching system is readily fixed with this inverted microscope with high speed camera. Prior to transferring the device with cells to the microscope suite, the microscope incubator is set ready at 37° C. and the CO₂ supply is directly connected to the stretching system. Both the stretching system computer and the microscope computer are synchronized to do the stretching and take time-lapse images. The images are acquired using a fluorescence objective lens with 20× and 40× magnification on an Olympus IX81 inverted microscope equipped with a charged coupled device monochrome camera (Qlmaging,) and DP71 software (Olympus, Japan). Images and figures are then processed and assembled with the Image J software (NIH, USA). FIG. 13 shows a photo image of the experimental setup, and FIG. 14 shows a snap-shot view of control software menu displaying a stretching graph of strength length vs time.

Stretching of Two Different Selected Cell Lines

The working capability and functionality of the I-MCS1 cell stretching system is tested using two different selected cell lines, namely NEB1-K14GFP, a wild type skin epithelial cells and the mutant cell line, NEB1-R125P.K14GFP. The object is to understand the effect of mechanical stress on these cells.

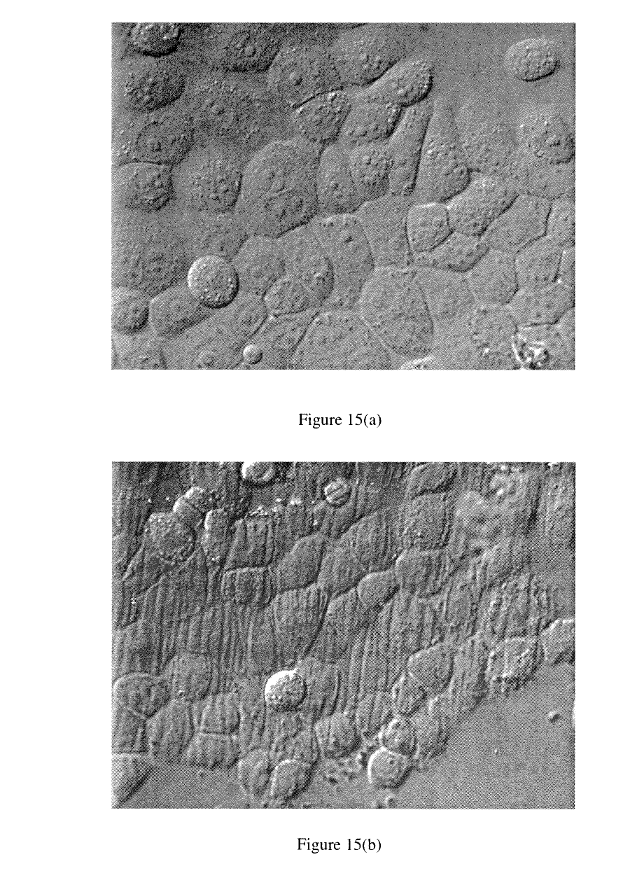

FIG. 15 shows phase contrast images of the wild-type NEB1.K14GFP cells which are grown on the fabricated device (a) before stretch, and (b) after subjecting to 30 mins of mechanical stress of 2 Hz and an amplitude of 50% using the I-MCS1 system.

It is observed that the wild-type cells start aligning perpendicular to the stretch after 30 mins of stretching using the I-MCS1 system.

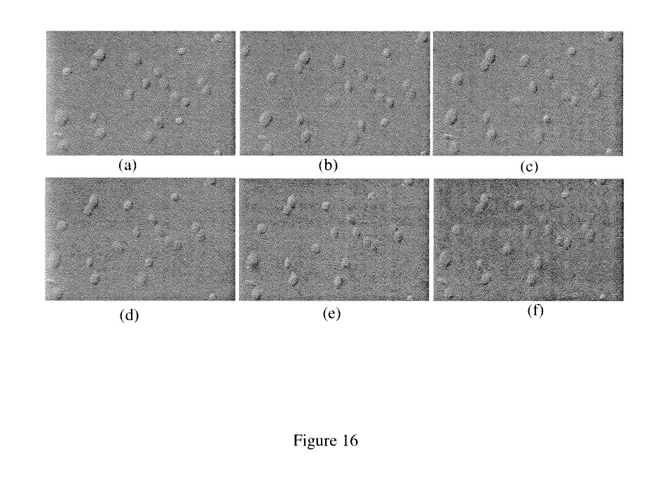

FIG. 16 shows phase contrast images of a separate sample of wild-typeNEB1.K14GFP cells which are grown on the fabricated device on which mechanical stretch is carried out at a frequency of 2 Hz and an amplitude of 50% for times varying up to 180 mins (more specifically, (a) at 0 min, (b) after 15 mins of stretching, (c) after 30 mins of stretching, (d) after 1 hour of stretching, (e) after 2 hours of stretching, and (f) after 3 hours of stretching) at 37° C. and 5% CO_{2.}

In FIG. 16, it is observed that the filaments appear to be disintegrating after 2 hours of stretching.

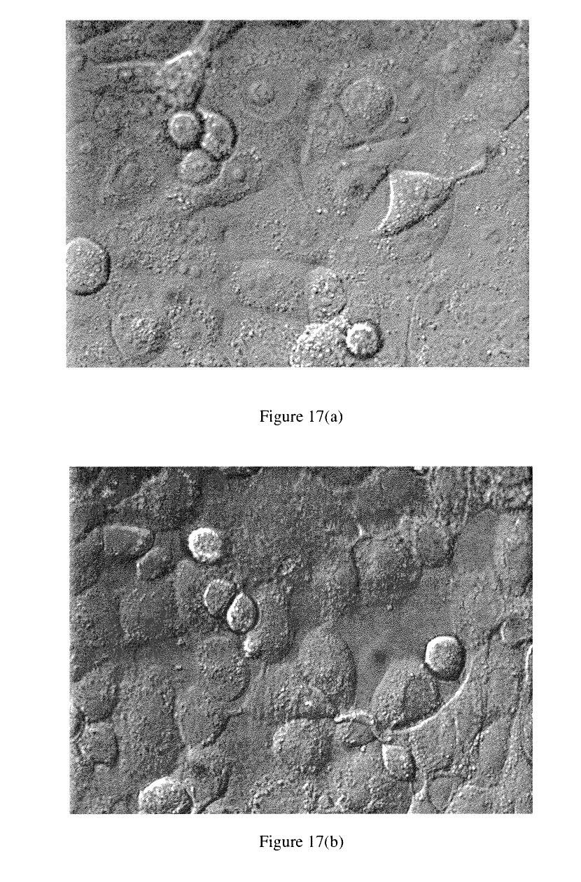

FIG. 17 shows phase contrast images of the mutant NEB1.K14GFP cells which are grown on the fabricated device (a) before stretch, and (b) after subjecting to 2 hours of mechanical stress or stretching using the I-MCS1 system.

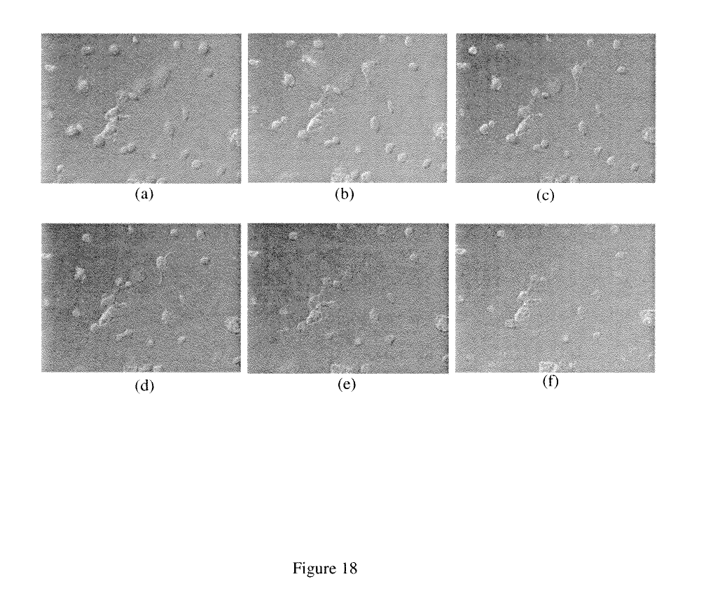

FIG. 18 shows phase contrast images of a separate sample of mutant NEB1.K14GFP cells which are grown on the fabricated device. The mutant NEB1.K14GFP cells are stretched for times varying up to 180 mins (more specifically, (a) at 0 min, (b) after 15 mins of stretching, (c) after 30 mins of stretching, (d) after 1 hour of stretching, (e) after 2 hours of stretching, and (f) after 3 hours of stretching) at 2 Hz with an amplitude of 50% at 37° C. and 5% CO₂. It is observed in both FIGS. 17 and 18 that the mutant cells are not able to withstand the mechanical stress after 15 mins of stretching. This suggests that the normal (e.g., wild type) epithelial cells can withstand mechanical stress up to 3 hours at 2 Hz with an amplitude of 50%, whereas the mutant cells of the corresponding type cannot withstand the mechanical stress even for about 15 mins under the same stretching conditions.

In the epidermis of patients with Epidermolysis bullosa simplex (EBS), keratinocytes break down in response to mechanical stress. This occurs because of the mutation of keratin intermediate filaments, which in some way renders the cells more susceptible to cytolysis on physical trauma. This fragility is studied by reproducing similar mechanical stress due to keratinocytes break down in the transgenic derived cell lines of NEB1.K14GFP and NEB1.R125P-K14GFP.

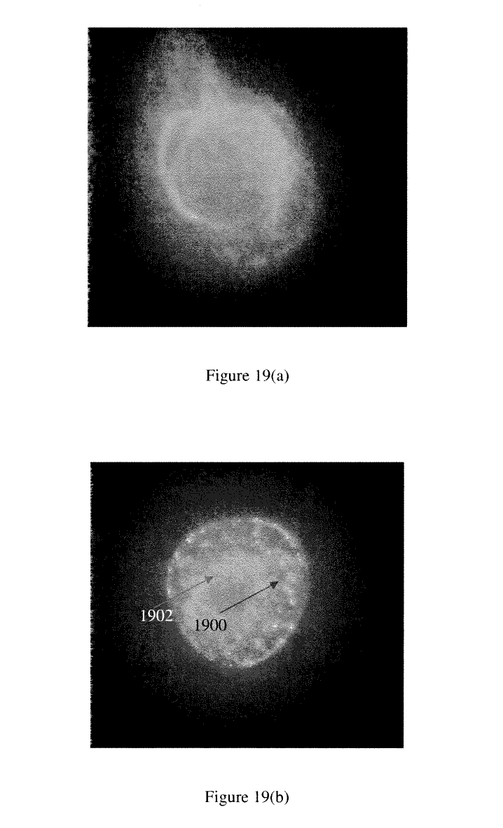

The changes in the keratin intermediate filament network in both wild-type and mutant keratin EBS cultured keratinocytes are observed. FIG. 19(a) shows an optical microscope image of a well-formed network of keratin filaments in the control wild-type NEB1.K14GFP cells before stretching.

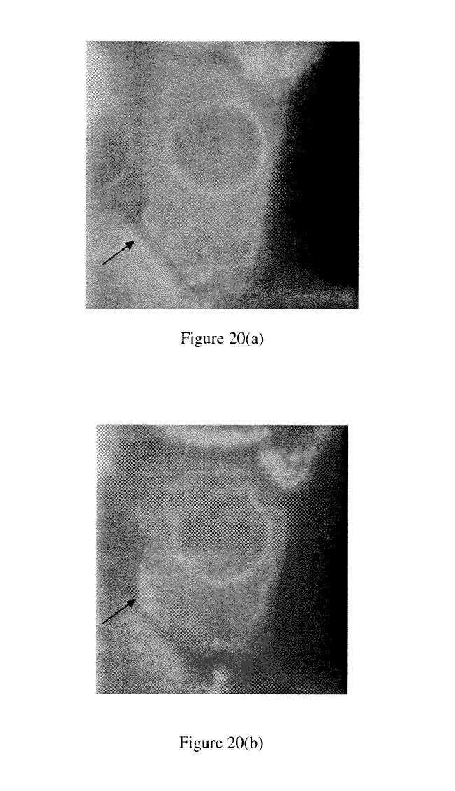

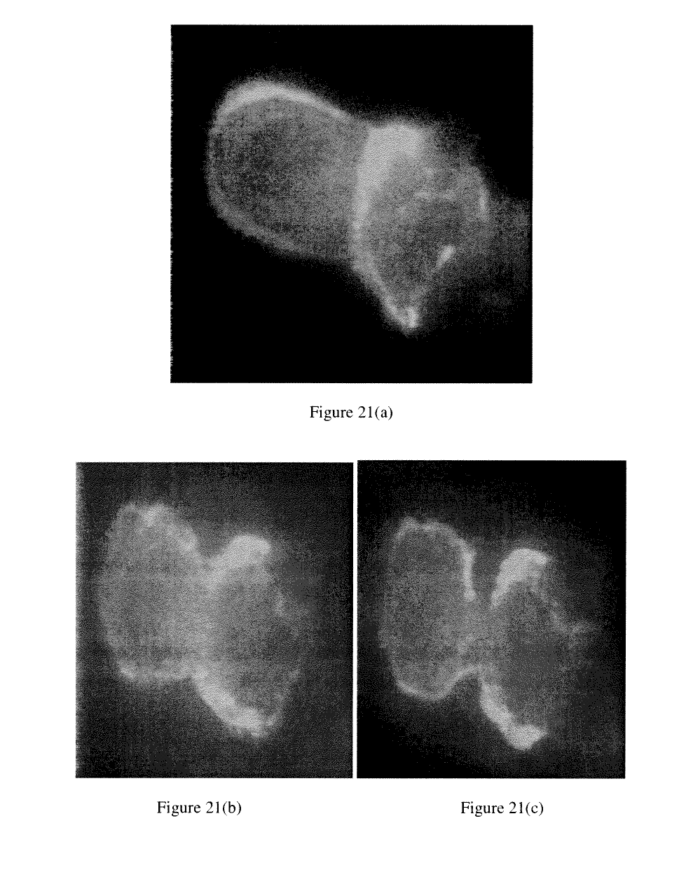

A mechanical stress at 4 Hz with an amplitude of 50% is applied. After 2 hour of stretching, it is observed that the keratin particles or aggregates appear spontaneously in places of close proximity to the cell periphery as indicated by an arrow 1900 in FIG. 19(b), and that the NEB1 cells start to exhibit thickening of filaments around the cell nucleus as indicated by an arrow 1902.

Whereas in the case of mutant cells as shown in FIG. 20(a), before stretch, clusters of keratin particles or aggregates appear spontaneously in places of close proximity to the cell periphery.

Keratin aggregates are diagnostic of Dowling-Meara EBS (Anton-Lamprecht and Schnyder, 1982) and are seen in situ in both intact and lysed cells nut, but not in every cell. After 5 mins of stretching or more specifically, cyclic stretching, it is observed in FIG. 20(b) that the appearance of keratin particles or aggregates in the mutant cells at the cell periphery suggesting that the mutant cells are not able to withstand the mechanical stress even for 5 mins, when applied with a mechanical stress of 4 Hz and an amplitude of 50% (c.f. parts indicated by the respective arrows in FIGS. 20(a) and 20(b)). The mutant cells show increased amounts of filament fragmentations, particularly along free edges.

FIG. 21 shows optical microscope images, or more specifically, fluorescence images of the breaking down of the cell junction of wild-type NEB1.K14GFP cells, when applied with a mechanical stress for (a) 0 min, (b) 1 hour, and (c) 2 hours, at 4 Hz with an amplitude of 50%.

As the time of stretching is increased, the cell-cell contacts become elongated, suggesting some elasticity in the cell-cell junctions (see FIG. 21(c)). This supports the hypothesis that keratin aggregation begins in areas of the cell rich in junctional proteins such as desmoplakin and that some intrinsic desmosomal elasticity may provide initial resistance to keratin fragmentation in response to stretch.

Mechanical stretch proves to be useful in analyses and evaluations of the function of intermediate filaments in tissues. The availability of various embodiments of a mechanical stretching device, which is able to reproduce at least part of the pathology of EBS in a tissue culture situation would be useful for analyzing the disease process and any hypothetical measures for disease symptoms of this disorder and related disorders thereof. The cell stretching experiments demonstrate an underlying need for much further analysis of the role of mechanical stress/forces in regulating biological responses at the cellular level.

Conclusively, the mechanical cell stretching system described herein is an easy-to-fabricate and easy-to-use system to apply mechanical stress on cells either for static or cyclic mode while applying the maximum stretching frequency of up to 10 Hz and the stretching length of up to 100%. Simultaneous live-cell stretching and imaging may also be performed using this system in order to study the effect of mechanical stress on live cells and capture the morphological changes taking place in the cells in response to stress. A real-time monitoring of cells may be possible using this system, when the cells are treated with drugs and to see the effect of drugs before and after applying the mechanical stress on cells in order to determine appropriate drugs to treat the mutant cells and to find out the optimum drug dosage.

While the invention has been particularly shown and described with reference to specific embodiments, it should be understood by those skilled in the art that various changes in form and detail may be made therein without departing from the spirit and scope of the invention as defined by the appended claims. The scope of the invention is thus indicated by the appended claims and all changes which come within the meaning and range of equivalency of the claims are therefore intended to be embraced.

1. A mechanical stretching device, comprising:
a stretchable substrate comprising at least one stretching material area within which a stretching material is placeable, and two engagement areas being located at opposite ends of the stretchable substrate, respectively;
two movable elements, each of which comprising an engagement portion, wherein each of the engagement portions is capable of engaging with one of the engagement areas; and
two motors, each of which being configured to drive one of the movable elements,
wherein the movable elements are movable by the motors such that the engagement portions cause, after having engaged with the engagement areas, either one end or both ends of the stretchable substrate to be stretched, wherein the ends of the stretchable substrate are to be stretched along opposite directions with respect to each other.

2. The stretching device according to claim 1,
wherein the movable elements are independently movable by the motors such that the ends of the stretchable substrate are independently stretchable.

3. The stretching device according to claim 1,
wherein the motors are motors which produce none or only little heat when in operation, in particular to maintain a physiological temperature.

4. The stretching device according to claim 3,
wherein the motors are voice coil motors.

5. The stretching device according to claim 1,
wherein the motors are drivable such that the stretchable substrate is continuously and/or periodically stretched and/or de-stretched.

6. The stretching device according to claim 5,
wherein the motors are drivable such that a stretching frequency or a de-stretching frequency of the stretchable substrate ranges from 0.5 Hz to 10 Hz or from 4 Hz to 10 Hz or from 0.5 Hz to 4 Hz or is 0.5 Hz or is 4 Hz or is 10 Hz.

7. The stretching device according to claim 1,
wherein the movement of the movable elements is controlled such that the stretchable substrate is stretched up to 100% in its length.

8. The stretching device according to claim 1,
wherein the motors are switchable between two driving modes, wherein, in a first driving mode, the stretchable substrate is caused to be stretched in opposite directions, whereas, in a second driving mode, the stretchable substrate is caused to be stretched in only one direction.

9. The stretching device according to claim 1,
further comprising a temperature controlling unit which controls the temperature of the stretchable substrate and its micro-environment to a predetermined temperature value.

10. The stretching device according to claim 1,
wherein the temperature controlling unit comprises a heating unit and/or a fan in particular for the uniform distribution of heat and to maintain the temperature.

11. The stretching device according to claim 1,
further comprising a humidity reservoir unit which controls or maintains the humidity of the environment of the stretchable substrate to a predetermined humidity value.

12. The stretching device according to claim 1,
further comprising a gas controlling unit which controls gas parameters of a gas surrounding the stretchable substrate to predetermined gas parameters.

13. The stretching device according to claim 12,
wherein the gas controlling unit comprises a gas inlet adapted to supply gas to the surrounding of the stretchable substrate.

14. The stretching device according to claim 1,
further comprising a gas-tight chamber surrounding the stretchable substrate, wherein the movable elements extend from outside of the chamber through walls of the chamber into the inside of the chamber, and wherein temperature, humidity and gas parameters within the chamber are controlled by the temperature controlling unit, and the gas controlling unit.

15. The stretching device according to claim 1,
further comprising an imaging device adapted to image the stretching material during the stretching of the stretchable substrate.

16. The stretching device according to claim 15,
wherein the imaging device comprises a microscope base plate.

17. The stretching device according to claim 15,
wherein the imaging device comprises a commercially available microscope.

18. The stretching device according to claim 1,
further comprising a motor controlling unit which controls the operation of the motors in a fully automated manner.

19. The stretching device according to claim 1,
wherein the stretchable substrate comprises, within the stretching material area, at least one stretching material well into which the stretching material is fillable.

20. The stretching device according to claim 1,
wherein the stretchable substrate comprises, within each engagement area, at least one engagement cavity into which the engagement portions of the moving elements are configured to be introduced.

21. The stretching device according to claim 1,
wherein the stretching material and/or the stretchable substrate are made of a stretchable material.

22. The stretching device according to claim 21,
wherein the stretchable material is a stretchable polymeric material.

23. The stretching device according to claim 22,
wherein the stretchable polymeric material is poly-di-methyl-siloxane (PDMS).

24. The stretching device according to claim 1,
wherein the stretching material and/or the stretchable substrate are coated with an adhesion layer facilitating binding of live cells to be analyzed in a stretch test carried out with the stretching device.

25. The stretching device according to claim 1,
wherein the stretching material and/or the stretchable substrate are coated with matrigel or collagen, such as collagen-IV, or fibronectin.

26. A stretching device, comprising:
a stretchable substrate placing area being configured to receive a stretchable substrate comprising: at least one stretching material area within which a stretching material is placeable, and two engagement areas being located at opposite ends of the stretchable substrate, respectively;
two movable elements, each of which comprising an engagement portion, wherein each of the engagement portions is capable of engaging with one of the engagement areas; and
two motors, each of which being configured to drive one of the movable elements,
wherein the movable elements are movable by the motors such that, when the stretchable substrate is placed within the stretchable substrate placing area, the engagement portions cause, after having engaged with the engagement areas, either one end or both ends of the stretchable substrate to be stretched, wherein the ends of the stretchable substrate are stretchable along opposite directions with respect to each other.

27. A method of carrying out a stretch test with live cells, the method comprising:
placing and cultivating live cells on a stretching material of a mechanical stretching device comprising:
a stretchable substrate comprising at least one stretching material area within which the stretching material is placeable, and two engagement areas being located at opposite ends of the stretchable substrate, respectively;
two movable elements, each of which comprising an engagement portion, wherein each of the engagement portions is capable of engaging with one of the engagement areas; and
two motors, each of which being configured to drive one of the movable elements,
wherein the movable elements are movable by the motors such that the engagement portions cause, after having engaged with the engagement areas, either one end or both ends of the stretchable substrate to be stretched, wherein the ends of the stretchable substrate are to be stretched along opposite directions with respect to each other.

28. The method according to claim 27, wherein the stretchable substrate of the stretching device is imaged with the imaging device while stretching the stretchable substrate.

29. The method according to claim 27, wherein the stretchable substrate of the stretching device is imaged with the imaging device while stretching the stretchable substrate by holding the substrate in a pre-determined stretched position for a pre-determined period of time.

30. The method according to claim 27, wherein the cells are eukaryotic cells.

31. The method according to claim 30, wherein the cells are selected from the group consisting of epithelial cells, muscle cells, cells isolated from intestines, cells isolated from airways, cells derived from bone, cells derived from cartilage and cells isolated from blood stream.

32. The method according to claim 27, wherein the cells are selected from the group consisting of cardiac myocytes, endothelial muscle cells, smooth muscle cells, skeletal muscle cells, connective tissue fibroblasts and epidermal keratinocytes.

33. The method according to claim 27, wherein the live cells are wild type cells or genetically modified mutant cells.
